(19) United States
(12) Patent Application Publication
(10) Pub. No.: US 2025/0334196 A1
(43) Pub. Date: Oct. 30, 2025

(54) AIR INFLATION DEVICE

(71) Applicant: TUNG CHENG CHEN, Taichung City (TW)

(72) Inventor: TUNG CHENG CHEN, Taichung City (TW)

(21) Appl. No.: 18/651,240

(22) Filed: Apr. 30, 2024

(51) Int. Cl. F16K 24/00; A61F 5/01

(52) U.S. Cl. CPC F16K 24/00; A61F 5/0111; A61F 5/012

(57) ABSTRACT

An air inflation device has a body, two switching valves, and a deflation valve. The body has an interior space formed therein, two outlets formed thereon at a spaced interval, and a pumping part. The pumping part is elastic and is pressable to compress air in the interior space to flow toward the two outlets. Each one of the two switching valves is disposed to the body and is operable to communicate with the interior space and a respective one of the two outlets. The deflation valve is disposed to the body and is operable to allow the interior space and an exterior space of the body to communicate with each other. The two switching valves allow multiple objects to be all or partially inflated or deflated at a time by operating the pumping part or the deflation valve.

### BACKGROUND OF THE INVENTION

### 1. Field of the Invention

The present invention relates to air inflation, and particularly to an air inflation device.

### 2. Description of Related Art

A conventional air pump assembly applied on a pneumatic walker normally has a simple air pump made of an elastic material such as rubber. The air pump is connected to an airbag of the pneumatic walker and is pressable to inflate the airbag. During deflation, the air bag is deflated by another component of the conventional air pump assembly such as a deflation valve. Thereby, a space inside the pneumatic walker surrounded by the air bag is adjustable.

However, the conventional air pump assembly lacks the ability of air distribution. Specifically, when the conventional air pump assembly inflates or deflates multiple objects, the multiple objects are all inflated or deflated but cannot be partially or individually inflated or deflated at a time. Thereby, range of application of the conventional air pump assembly is thus limited.

To overcome the shortcomings of the conventional air pump assembly, the present invention tends to provide an air inflation device to mitigate or obviate the aforementioned problems.

### SUMMARY OF THE INVENTION

The main objective of the present invention is to provide an air inflation device which allows multiple objects to be all or partially inflated or deflated at a time.

The air inflation device has a body, two switching valves, and a deflation valve. The body has a pumping part, an interior space, and two outlets. The interior space is formed in the body, and the two outlets are formed on the body at a spaced interval. The pumping part is elastic and is pressable to compress air in the interior space to flow toward the two outlets. The two switching valves are disposed to the body, and each one of the two switching valves is operable to communicate with the interior space and a respective one of the two outlets. The deflation valve is disposed to the body and is operable to allow the interior space and an exterior space of the body to communicate with each other.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a preferred embodiment of an air inflation device in accordance with the present invention;

FIG. 2 is an exploded view of the air inflation device in FIG. 1 with a body of the air inflation device shown in a sectional view;

FIG. 3 is a sectional side view of the air inflation device in FIG. 1;

FIG. 4 is an operational sectional side view of the air inflation device in FIG. 1;

FIG. 5 is an enlarged view of FIG. 4;

FIGS. 6 and 7 are operational sectional side views of a switching valve of the air inflation device in FIG. 1;

24 FIG. 8 is an operational sectional side view of a deflation valve of the air inflation device in FIGS. 1; and

FIG. 9 is a perspective view of the air inflation device in FIG. 1 applied to a pneumatic walker.

### DETAILED DESCRIPTION OF THE PREFERRED EMBODIMENT

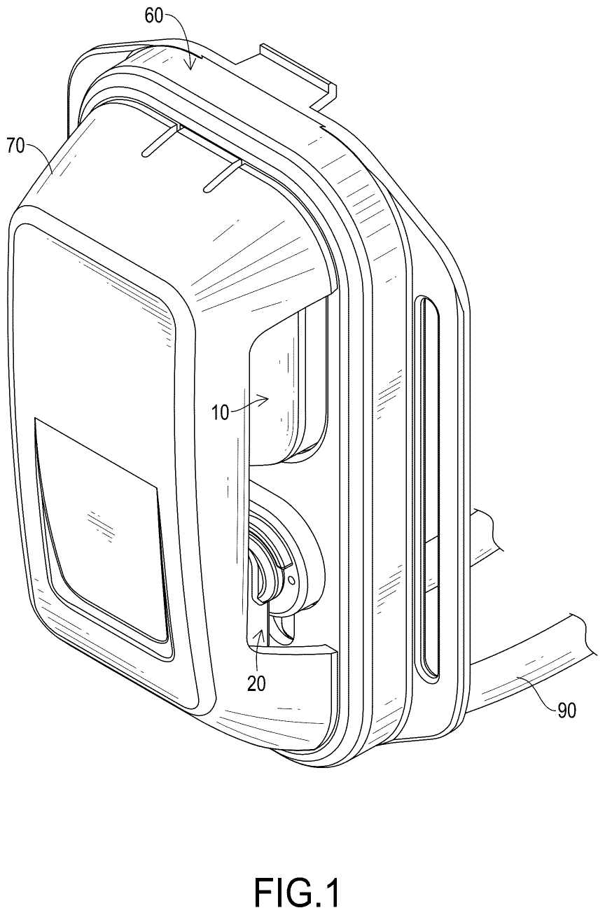

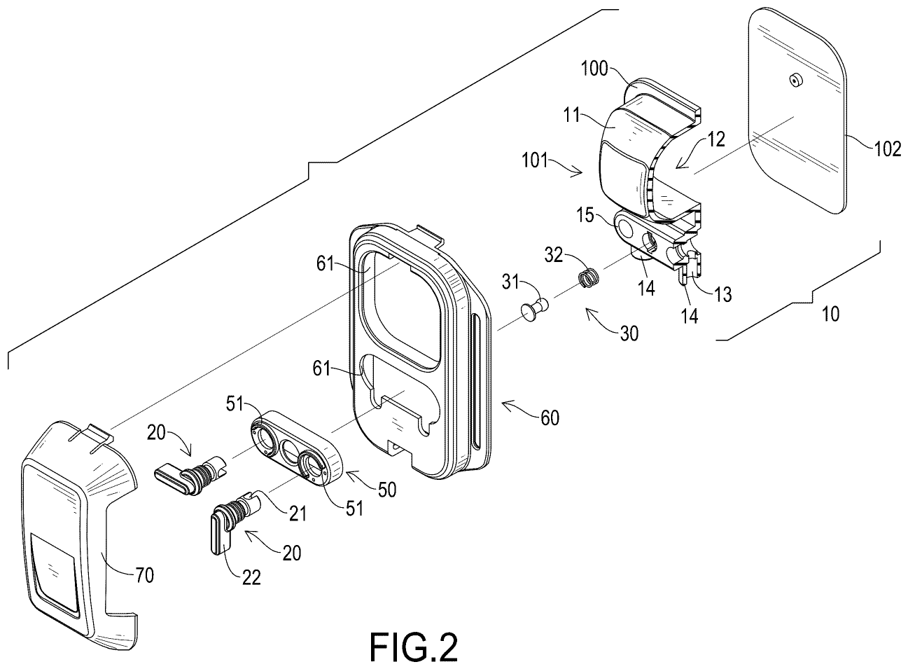

With reference to FIGS. 1 and 2, a preferred embodiment of an air inflation device in accordance with the present invention has a body 10, two switching valves 20, and a deflation valve 30. The two switching valves 20 are disposed to the body 10 for controlling air distribution during inflation and deflation, and the deflation valve 30 is disposed to the body 10 for deflation.

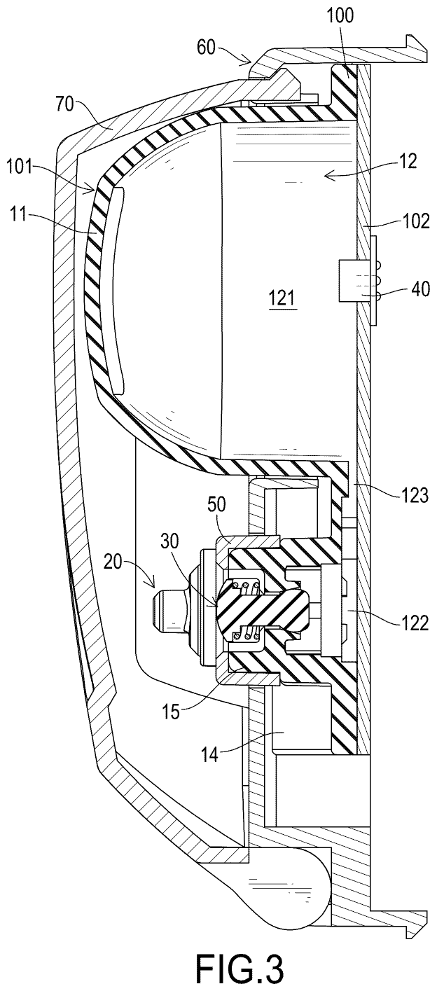

With reference to FIGS. 2 and 3, the body 10 has a pumping part 11, an interior space 12, and two outlets 13. The interior space 12 is formed in the body 10, and the two outlets 13 are formed on a bottom of the body 10 and arranged at a spaced interval. The pumping part 11 is elastic and is pressable to compress air in the interior space 12 to flow toward the two outlets 13.

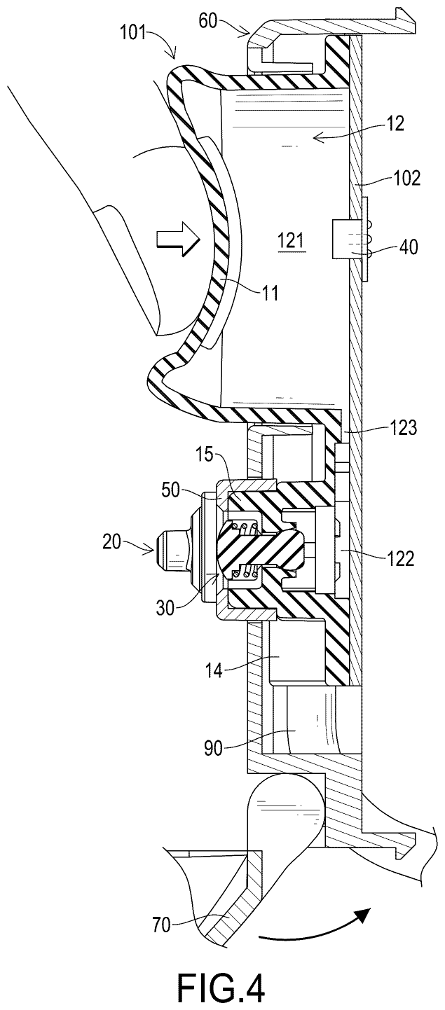

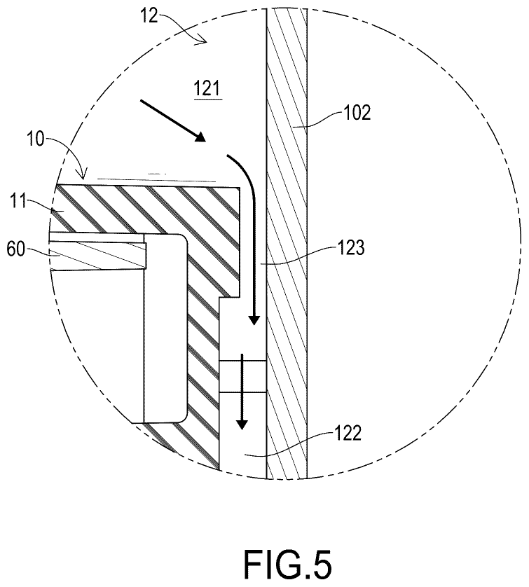

With reference to FIG. 3, in the preferred embodiment, the interior space 12 specifically has an inflation section 121, an outlet section 122, and a channel 123. The inflation section 121 is formed in the pumping part 11, and the outlet section 122 is spaced from the inflation section 121. The channel 123 extends from the inflation section 121 to the outlet section 122. With reference to FIGS. 4 and 5, when the pumping part 11 is pressed, the air in the inflation section 121 is compressed to flow through the channel 123 to the outlet section 122. The channel 123 may have a check valve or the like disposed therein to prevent air from reflowing, which is conventional and is not particularly shown in the drawings.

With reference to FIGS. 2 and 3, in the preferred embodiment, the body 10 has a first casing 101 and a second casing 102. The first casing 101 has a flange 100, the pumping part 11, two bottom protrusions 14, and a valve mount 15. The pumping part 11, the two bottom protrusions 14, and the valve mount 15 protrude from a side of the flange 100 along a same direction, and the second casing 102 is connected to the other side of the flange 100. The valve mount 15 and the two bottom protrusions 14 are spaced from the pumping part 11, and the two bottom protrusions 14 are connected to the valve mount 15.

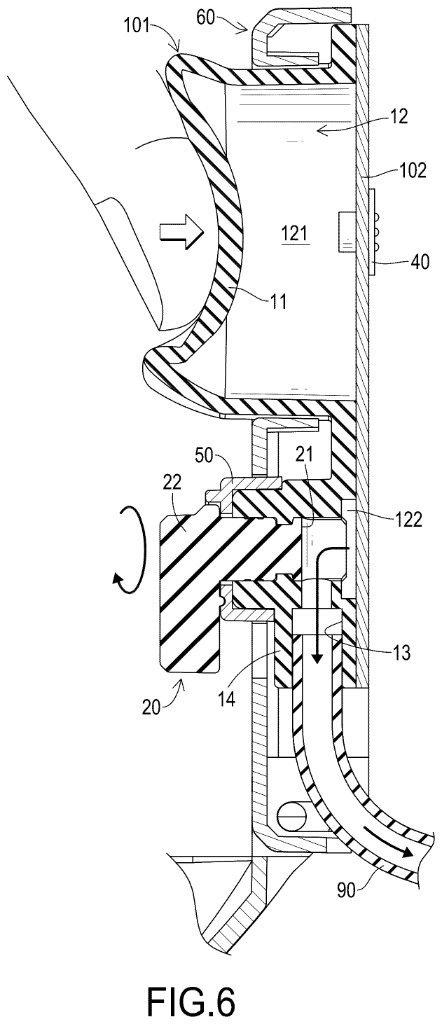

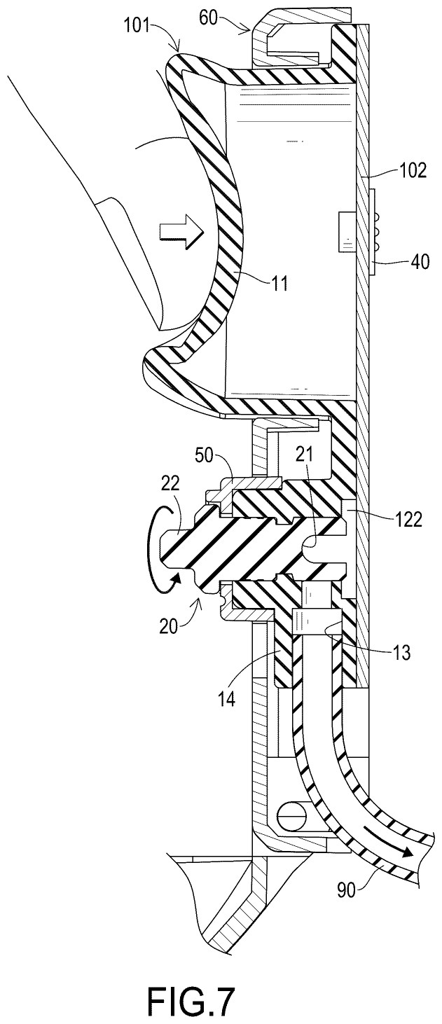

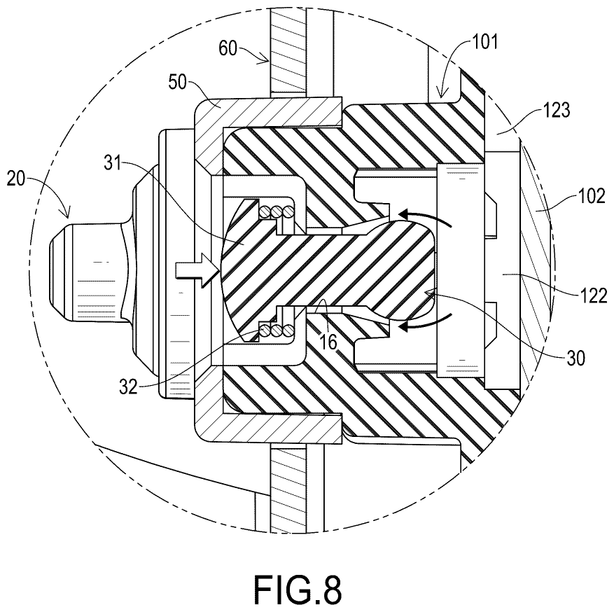

With reference to FIGS. 2 and 3, in the interior space 12, the inflation section 121 is enclosed by the pumping part 11 and the second casing 102, and the outlet section 122 is enclosed by the valve mount 15 and the second casing 102. The channel 123 is formed between the body 10 and the second casing 102. The two outlets 13 are respectively formed on the two bottom protrusions 14, and with reference to FIGS. 6 and 7, each one of the two outlets 13 can have a respective hose 90 installed thereon for connecting an object for inflation or deflation.

Additionally, with reference to FIGS. 2 and 3, a check valve 40 is disposed on the body 10. When the pumping part 11 is pressed, the pumping part 11 shrinks, and the air in the inflation section 121 is compressed to flow to the channel 123. After pressing of the pumping part 11 ends, the check valve 40 allows air outside the body 10 to flow therethrough and into the inflation section 121 such that the pumping part 11 expands and is able to be pressed again. Specifically, the check valve 40 is disposed on the second casing 102.

Additionally, the valve mount 15 has two holes formed thereon at a spaced interval for the two switching valves 20 to be mounted therethrough, which improves the stability of the two switching valves 20 disposed to the body 10. Also, the valve mount 15 allows the deflation valve 30 to be mounted thereon for deflation.

Further, in the preferred embodiment, the first casing 101 is made of an elastic material. Thereby, the valve mount 15 on the first casing 101 allows the holes for mounting the two switching valves 20 to be designed smaller, which allows the two switching valves 20 to be tightly mounted to the valve mount 15.

With reference to FIGS. 6 and 7, specifically, each one of the two switching valves 20 is mounted through said hole on the valve mount 15 and extends into the outlet section 122 of the interior space 12. Each one of the two switching valves 20 is operable to communicate with the interior space 12 and a respective one of the two outlets 13.

In the preferred embodiment, each one of the two switching valves 20 is rotatably mounted through the valve mount 15 and has a passageway 21 formed thereon. The passageway 21 is recessed on an end of said switching valve 20 extending into the outlet section 122. With reference to FIGS. 6 and 7, by rotating said switching valve 20, the passageway 21 may be aligned with the corresponding outlet 13 to communicate with the outlet section 122 of the interior space 12 and the corresponding outlet 13 or deflected from the corresponding outlet 13 to stop the air from flowing to the corresponding outlet 13 from the outlet section 122.

Further, with reference to FIGS. 2, 6, and 7, each one of the two switching valves 20 has a trigger 22 formed on the other end of said switching valve 20 protruding from the body 10. Thereby, each one of the two switching valves 20 can be rotated by pushing the trigger 22, which improves convenience in operation. Further, the trigger 22 and the passageway 21 extend in a same direction, which allows the two switching valves 20 to inform a user of a direction of the passageway 21 inside the body 10 by the trigger 22 outside the body 10.

Additionally, with reference to FIGS. 2, 6, and 7, the air inflation device further has a saddle 50 mounted on the valve mount 15 of the body 10 and having two limiting protrusions 51. The two switching valves 20 are sequentially mounted through the saddle 50 and the valve mount 15. When operated to rotate the corresponding switching valve 20, the trigger 22 abuts against a respective one of the two limiting protrusions 51 if the corresponding switching valve 20 over-rotates. Each one of the two limiting protrusions 51 is formed to restrict a rotation range of the trigger 22 of the corresponding switching valve 20 and prevent the trigger 22 from collision.

With reference to FIGS. 2, 3, and 8, as described above, the deflation valve 30 is mounted on the valve mount 15 of the body 10. Specifically, the valve mount 15 further has a deflation hole 16 formed thereon and between the two holes for mounting the two switching valves 20, and the deflation hole 16 communicates with the outlet section 122 of the interior space 12 and an exterior space of the body 10. The deflation valve 30 has a plug 31 mounted to the deflation hole 16 and is movable for deflation.

With reference to FIGS. 8, when the plug 31 moves inward toward the second casing 102, the plug 31 forms an annular gap between the plug 31 and a peripheral surface surrounding the deflation hole 16 for air inside the interior space 12 to flow outward; when the plug 31 moves outward away from the second casing 102, the plug 31 obstructs the deflation hole 16 to prevent air from flowing through the deflation hole 16 from the outlet section 122 of the interior space 12.

In the preferred embodiment, the deflation valve 30 further has a spring 32 disposed between the plug 31 and the valve mount 15 of the body 10. The spring 32 provides an elastic force to keep the plug 31 obstructing the deflation hole 16, which improves airtightness and prevents air leakage. Still, the plug 31 can be pushed and compress the spring 32 to move for deflation.

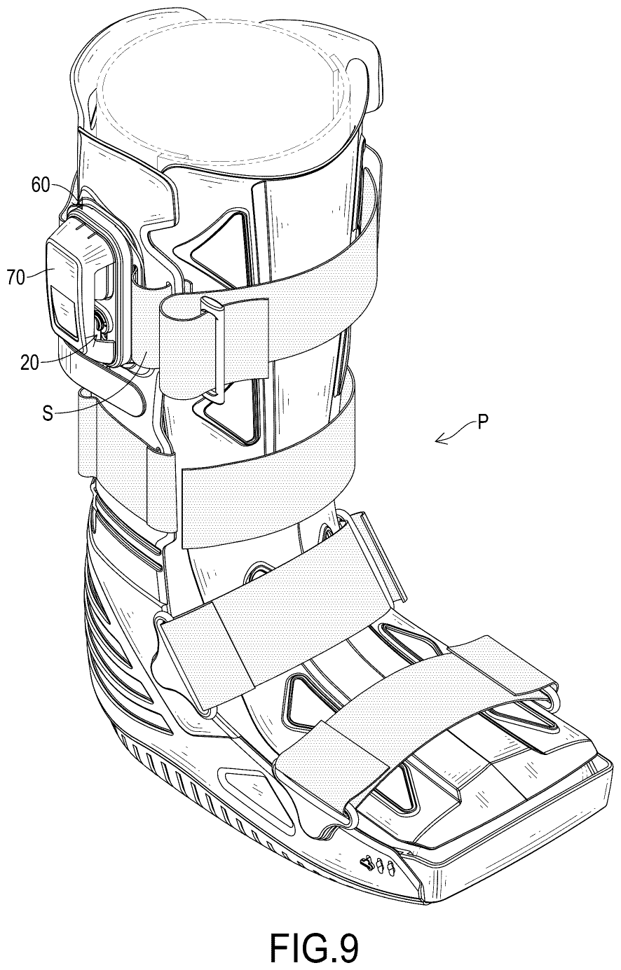

With reference to FIG. 9, the air inflation device may be applied in a pneumatic walker P. The air inflation device can be installed on a side of the pneumatic walker P. Specifically, with reference to FIG. 2, the air inflation device has a frame 60. The frame 60 has two openings 61 respectively matching outer profiles of the pumping part 11 and the valve mount 15 of the body. After placing the body 10, the frame 60 is placed to limit the body 10 between the frame 60 and the pneumatic walker P and to allow the pumping part 11 and the valve mount 15 to be respectively mounted through the two openings, and the frame 60 is fixed on the side of the pneumatic walker P by a strap S. In different application, the body 10 may still be secured by the frame 60.

The pneumatic walker P has two or more airbags surrounding an adjustable space and respectively communicating with the two outlets 13 of the body via connections of the hoses 90 installed to the two outlets 13. After a user wears the pneumatic walker P over an injured foot, the two switching valves 20 may be opened or closed first. Afterwards, when the pumping part 11 of the body 10 is pressed, the air in the interior space 12 flows through the opened switching valve(s) 20 and the corresponding hose(s) 90 to the corresponding airbag(s), which allows the airbags to be all or partially inflated at a time.

The two switching valves 20 can also be opened or closed before deflation, and then the deflation valve 30 is operated. After the plug 31 is pushed to form the gap for deflation, air pressure inside the outlet section 122 of the interior space 12, the opened switching valve(s) 20, the corresponding hose(s) 90, and the corresponding airbag(s) drives the air inside these components to flow outside through the gap, which allows the airbags to be all or partially deflated at a time. Thus, the user can precisely adjust the space surrounded by the airbags according to injured portions by inflating or deflating the airbags flexibly.

With reference to FIGS. 2 to 4, the air inflation device further has a covering 70 pivotally connected to the frame 60. When the air bags of the pneumatic walker P need not be inflated or deflated, the covering 70 pivots relative to the frame 60 to cover the pumping part 11 of the body 10 and the two switching valves 20, which prevents inadvertent inflation or deflation. When the air bags need to be inflated or deflated, the covering 70 can pivot relative to the frame 60 with reference to FIG. 4 to expose the pumping part 11 and the two switching valves 20 for operation.

With the two switching valves 20, the air inflation device in accordance with the present invention provides a utility of air distribution and allows multiple objects to be all or partially inflated or deflated at a time. Thus, the air inflation device has high flexibility in operation and can have wider application compared to the conventional air pump assembly.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. An air inflation device comprising:
a body having
an interior space formed in the body; and
two outlets formed on the body at a spaced interval; and
a pumping part being elastic and pressable to compress air in the interior space to flow toward the two outlets;
two switching valves disposed to the body, and each one of the two switching valves being operable to communicate with the interior space and a respective one of the two outlets; and
a deflation valve disposed to the body and being operable to allow the interior space and an exterior space of the body to communicate with each other.

2. The air inflation device as claimed in claim 1, wherein
the interior space has
an inflation section formed in the pumping part;
an outlet section; and
a channel extending from the inflation section to the outlet section and configured for air to flow from the inflation section to the outlet section; and
each one of the two switching valves is operable to communicate with the outlet section of the interior space and the corresponding outlet.

3. The air inflation device as claimed in claim 1, wherein
the body has a deflation hole formed thereon and communicating with the interior space and the exterior space of the body; and
the deflation valve has a plug disposed to the deflation hole and being movable to obstruct the deflation hole or form a gap between the plug and a peripheral surface surrounding the deflation hole.

4. The air inflation device as claimed in claim 2, wherein
the body has a deflation hole formed thereon and communicating with the outlet section of the interior space and the exterior space of the body; and
the deflation valve has a plug disposed to the deflation hole and being movable to obstruct the deflation hole or form a gap between the plug and a peripheral surface surrounding the deflation hole.

5. The air inflation device as claimed in claim 3, wherein the deflation valve has a spring disposed between the plug and the body and configured to provide an elastic force to keep the plug obstructing the deflation hole.

6. The air inflation device as claimed in claim 4, wherein the deflation valve has a spring disposed between the plug and the body and configured to provide an elastic force to keep the plug obstructing the deflation hole.

7. The air inflation device as claimed in claim 2, wherein
the body has
a first casing having a flange, the pumping part, and a valve mount; and
a second casing connected to the flange;
the pumping part and the valve mount protrude from the flange;
the inflation section of the interior space is enclosed by the pumping part and the second casing; and
the outlet section of the interior space is enclosed by the valve mount and the second casing.

8. The air inflation device as claimed in claim 7, wherein the air inflation device has a frame having
two openings configured for the pumping part and the valve mount to be mounted therethrough to position the body.

9. The air inflation device as claimed in claim 8, wherein the air inflation device has a covering pivotally connected to the frame and configured to cover the pumping part and the two switching valves as the covering pivots.

10. The air inflation device as claimed in claim 1, wherein the air inflation device has a check valve disposed on the body and configured for air to flow therethrough and into the interior space.

11. The air inflation device as claimed in claim 1, wherein each one of the two switching valves is rotatably disposed on the body and has
a passageway configured to be aligned with the corresponding outlet to connect the interior space and the corresponding outlet by rotating said switching valve.

12. The air inflation device as claimed in claim 2, wherein each one of the two switching valves is rotatably disposed on the body, extends into the outlet section of the interior space, and has
a passageway configured to be aligned with the corresponding outlet to connect the outlet section and the corresponding outlet by rotating said switching valve.

13. The air inflation device as claimed in claim 7, wherein each one of the two switching valves is rotatably mounted through the valve mount of the body, extends into the outlet section of the interior space, and has
a passageway configured to be aligned with the corresponding outlet to connect the outlet section and the corresponding outlet by rotating said switching valve.

14. The air inflation device as claimed in claim 11, wherein each one of the two switching valves has a trigger formed on an end of said switching valve protruding from the body.

15. The air inflation device as claimed in claim 12, wherein each one of the two switching valves has a trigger formed on an end of said switching valve protruding from the body.

16. The air inflation device as claimed in claim 13, wherein each one of the two switching valves has a trigger formed on an end of said switching valve protruding from the body.

17. The air inflation device as claimed in claim 14, wherein
the air inflation device has a saddle disposed on the body and having two limiting protrusions;
each one of the two switching valves is rotatably mounted through the saddle and to the body; and
the trigger of each one of the two switching valves is configured to abut against a respective one of the two limiting protrusions of the saddle so as to restrict a range of rotation of the trigger.

18. The air inflation device as claimed in claim 15, wherein
the air inflation device has a saddle disposed on the body and having two limiting protrusions;
each one of the two switching valves is rotatably mounted through the saddle and to the body; and
the trigger of each one of the two switching valves is configured to abut against a respective one of the two limiting protrusions of the saddle so as to restrict a range of rotation of the trigger.

19. The air inflation device as claimed in claim 16, wherein
the air inflation device has a saddle disposed on the body and having two limiting protrusions;
each one of the two switching valves is rotatably mounted through the saddle and to the body; and
the trigger of each one of the two switching valves is configured to abut against a respective one of the two limiting protrusions of the saddle so as to restrict a range of rotation of the trigger.
